(19) United States
(12) Patent Application Publication
(10) Pub. No.: US 2019/0010530 A1
(43) Pub. Date: Jan. 10, 2019

(54) METHODS AND COMPOSITIONS FOR NUCLEIC ACID ASSEMBLY

(71) Applicant: Gen9, Inc., Boston, MA (US)

(72) Inventor: Ishtiaq Saaem, Chelsea, MA (US)

(73) Assignee: Gen9, Inc., Boston, MA (US)

(21) Appl. No.: 16/064,044

(22) PCT Filed: Dec. 21, 2016
(86) PCT No.: PCT/US2016/067927

§ 371 Date: Jun. 20, 2018

Related U.S. Application Data
(60) Provisional application No. 62/270,131, filed on Dec. 21, 2015.

(51) Int. Cl. C12P 19/34; C12N 15/66; C12N 15/10

(52) U.S. Cl. CPC C12P 19/34; C40B 40/06; C12N 15/1093; C12N 15/66

(57) ABSTRACT

Aspects of the disclosure relate to compositions and methods for polynucleotide assembly. In some embodiments, a terminator oligonucleotide is provided.

### CROSS-REFERENCE TO RELATED APPLICATIONS

This application claims the benefit of and priority to U.S. Provisional Patent Application No. 62/270,131 file Dec. 21, 2015, the entire disclosure of which is incorporated herein by reference.

### FIELD

The present disclosure relates to compositions and methods for in vitro nucleic acid synthesis and assembly.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids can also be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids may also be used as therapeutic compositions (e.g., for modifying gene expression, for gene therapy, etc.) or as diagnostic tools (e.g., as probes for disease conditions, etc.).

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids that can be used in research, industry, agriculture, and/or medicine. However, one limitation of currently available assembly techniques is the relatively high error rate and failure to assemble certain genes (due to, e.g., high GC content or difficulty in parsing). As such, a need exists for improved assembly methods.

### SUMMARY

One aspect of the disclosure relates to a non-naturally occurring nucleic acid sequence comprising a Y—X—Z—O stem-loop, wherein:
a. Y is a nucleotide sequence of 5 to 30 nucleotides in length;
b. X is a nucleotide sequence of 3 to 12 nucleotides in length, each nucleotide therein not base pairing with any other nucleotide within X when Y and Z form a stem;
c. Z is a nucleotide sequence of 5 to 50 nucleotides in length and having at least 70% complementarity to Y; and
d. O is either absent or an overhang protruding from the stem formed between Y and Z.

In some embodiments, X forms a loop. It should be noted that Y can be 5′ to X or 3′ to X. In other words, O can be a 5′ protrusion/overhang or a 3′ protrusion/overhang.

O may comprise a degenerate sequence (having, e.g., N degenerate positions that may or may not be contiguous wherein N is any integer). The nucleic acid sequence can include one or more dT-biotin. In one embodiment, the Y—X—Z portion has the sequence of SEQ ID NO.: 1.

Another aspect relates to a library of non-naturally occurring nucleic acid sequences, each member comprising a Y—X—Z—O stem-loop, wherein:
a. Y is a nucleotide sequence of 5 to 30 nucleotides in length;
b. X is a nucleotide sequence of 3 to 12 nucleotides in length, each nucleotide therein not base pairing with any other nucleotide within X when Y and Z form a stem;
c. Z is a nucleotide sequence of 5 to 50 nucleotides in length and having at least 70% complementarity to Y; and
d. O is a 5′ or 3′ overhang protruding from the stem formed between Y and Z and comprises a degenerate sequence having N degenerate positions;

wherein the library comprises at least 4^{N} members.

In some embodiments, each member of the library can contain one or more dT-biotin. All members, or a subset thereof, may have the same Y—X—Z. In one embodiment, the Y—X—Z portion of each member, or a subset of the library members, has the sequence of SEQ ID NO.: 1.

A further aspect relates to a method of modifying a nucleic acid molecule, comprising attaching the nucleic acid sequence (e.g., terminators) disclosed herein to the nucleic acid molecule. In some embodiments, the attaching step comprises ligating.

Still a further aspect relates to a method of assembling a target nucleic acid, comprising:
a. assembling a 5′ terminal construction oligonucleotide, at least one central construction oligonucleotides and a 3′ terminal construction oligonucleotide, wherein each construction oligonucleotide has two cohesive ends, at least one cohesive end being compatible with that of another construction oligonucleotide, such that when fully assembled in a predetermined order, the construction oligonucleotides form a target nucleic acid or a subconstruct thereof, wherein the 5′ terminal construction oligonucleotide has a 5′ primer binding site and the 3′ terminal construction oligonucleotide has a 3′ primer binding site;
b. attaching a terminator to an assembly product from step (a) at both ends, wherein the terminator has an overhang compatible with that of the assembly product; and
c. selectively amplifying a full assembly product using primers against the 5′ primer binding site and the 3′ primer binding site.

Yet another method of assembling a target nucleic acid is provided herein, comprising:
a. assembling a 5′ terminal construction oligonucleotide, at least one central construction oligonucleotides and a 3′ terminal construction oligonucleotide, wherein the at least one central construction oligonucleotides each has two cohesive ends each compatible with that of another construction oligonucleotide, such that when fully assembled in a predetermined order, the construction oligonucleotides form a target nucleic acid or a subconstruct thereof, wherein the 5′ terminal construction oligonucleotide has a 5′ blunt end and the 3′ terminal construction oligonucleotide has a 3′ blunt end;
b. attaching a terminator to a partial assembly product from step (a), wherein the terminator has an overhang compatible with that of the partial assembly product, and wherein the terminator comprises a label; and
c. removing the partial assembly product using a binding partner of the label.

In some embodiments, the 5′ terminal construction oligonucleotide has a 5′ primer binding site and the 3′ terminal construction oligonucleotide has a 3′ primer binding site, wherein the method further comprising amplifying a full assembly product using primers against the 5′ primer binding site and the 3′ primer binding site. In certain embodiments, to facilitate removal of the partial assembly product, the label can be biotin and the binding partner can be one or more of avidin, streptavidin and NeutrAvidin.

### BRIEF DESCRIPTION OF THE FIGURES

FIG. 1 illustrates an exemplary use of terminator oligonucleotides during subassembly of subconstructs.

FIGS. 2A-2B illustrate an exemplary use of terminator oligonucleotides during assembly of target.

FIG. 3 illustrates an exemplary use of biotin-terminator oligonucleotides during subassembly.

FIG. 4 illustrates an exemplary design of terminator oligonucleotides with and without biotin.

FIG. 5 illustrates an exemplary assembly strategy.

FIG. 6 illustrates an exemplary terminator oligonucleotide.

### DETAILED DESCRIPTION OF THE DISCLOSURE

Aspects of the disclosure relate to compositions and methods for the assembly of nucleic acid molecules. Aspects of the disclosure further relate to compositions and methods for assembling a polynucleotide (e.g., a target nucleic acid or a subassembly intermediate) from oligonucleotides (e.g., construction oligos or subconstructs) using hairpin-containing terminator oligonucleotides. In some embodiments, the terminator oligonucleotides disclosed herein can improve assembly efficiency and/or accuracy, and can help remove undesirable or incorrect assembly products.

To assemble a target nucleic acid, one strategy is to analyze the sequence of the target nucleic acid and parse it into two or more construction oligonucleotides having compatible (e.g., complementary) cohesive ends between one another, so that together the two or more construction oligonucleotides can be assembled (e.g., ligated) into the target nucleic acid. Assembly can be carried out using hierarchical, sequential and/or one-step assembly. By way of example only, hierarchical assembly of oligonucleotides A, B, C and D (each a construction oligonucleotide) into a A+B+C+D target may include assembling A+B and C+D first (each a subconstruct or subassembly), then A+B+C+D. Sequential assembly may include assembling A+B (a primary subconstruct or subassembly), then A+B+C (a secondary subconstruct or subassembly), and finally A+B+C+D (target). One-step assembly combines A, B, C and D in one reaction to produce A+B+C+D. It should be noted that different strategies can be mixed where a portion of the construction oligonucleotides are assembled using one strategy while another portion a different strategy.

The construction oligonucleotides can be chemically synthesized, e.g., on a solid support as described in more detail below. In some embodiments, the construction oligonucleotides can be synthesized in sufficient amount so as to enable direct subassembly or total assembly without the need to amplify one or more of the construction oligonucleotides. In certain embodiments, the construction oligonucleotides, after chemical synthesis, may be first subject to subassembly into subconstructs, which can be amplified (e.g., in a polymerase based reaction) and then subject to further assembly into secondary subconstructs or the final target. In some embodiments, one or more construction oligonucleotides can be amplified before assembly.

To facilitate amplification, one or more construction oligonucleotides and/or subconstructs may be designed to comprise one or more primer biding sites to which a primer can bind or anneal in a polymerase chain reaction. The primer biding sites can be designed to be universal (i.e., the same) to all construction oligonucleotides or a subset thereof, or two or more subconstructs. Universal primer biding sites (and corresponding universal primers) can be used to amplify all construction oligonucleotides or subconstructs having such universal primer biding sites in a polymerase chain reaction. Primer binding sites that are specific to one or more select construction oligonucleotides and/or subconstructs can also be designed, so as to allow targeted, specific amplification of the select construction oligonucleotides and/or subconstructs. In some embodiments, one or more construction oligonucleotides and/or subconstructs may contain nested or serial primer binder sites at one or both ends where one or more outer primers and inner primers can bind. In one example, the construction oligonucleotides and/or subconstructs each have binding sites for a pair of outer primers and a pair of inner primers. One or both of the pair of outer primers may be universal primers. Alternatively, one or both of the pair of outer primers may be unique primers. In some embodiments, before assembly, each of the construction oligonucleotides is individually amplified. The construction oligonucleotides can also be pooled into one or more pools for amplification. In one example, all of the construction oligonucleotides are amplified in a single pool. In certain embodiments, the amplified construction oligonucleotides are assembled via polymerase based assembly or ligation. The amplified construction oligonucleotides may be assembled hierarchically or sequentially or in a one-step reaction into the target nucleic acid.

One or more of the primer binding sites can be designed to be part of the construction oligonucleotides that are incorporated into the final target nucleic acid. In some embodiments, all or part of each primer binding site can be in the form of a flanking region outside the central portion of a construction oligonucleotide, wherein the central portion is incorporated into the final target nucleic acid and the flanking region needs be removed before assembly. To that end, one or more restriction sites can be designed to allow removal of the flanking region.

One or more of the above steps can be facilitated by the use of terminator oligonucleotides. For example, during subassembly and/or assembly, incomplete subassembly or assembly products (e.g., where one or more construction oligonucleotides are missing from the subconstruct or final construct) may be present. These incomplete products are difficult to remove or separate from the completely and correctly assembled products due to proximity in size. In subsequence polymerase based amplification, the incomplete molecules are often amplified along with the complete assembly products. To suppress amplification of the incomplete products, terminator oligonucleotides can be attached to one or both ends of the subassembly or assembly products, such that amplification of the incomplete products is disfavored without affecting amplification of complete assembly products. The terminator oligonucleotides may contain one or more labels such as biotin and/or digoxigenin that can be bound by its binding partner for subsequent removal. This way, the correct assembly products can be enriched and/or purified.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

“Assembly” means a process in which short DNA sequences (construction oligonucleotides) are attached in a particular order to form a longer DNA sequence (target). “Subassembly” means an intermediate step or product where a subset of the construction oligonucleotides are attached to form a subconstruct that is a portion of the final target.

“CEL” or “cohesive end ligation” refers to the process of joining DNA fragments in a predesigned order using cohesive ends that are at least partially complementary to one another. The cohesive ends can be generated by restriction enzyme digestion or can be directly synthesized on a solid support.

As used herein, a “chip” refers to a DNA microarray with many oligonucleotides attached to a planar surface. The oligonucleotides on a chip can be any length. In some embodiments, the oligonucleotides are about 200 nucleotides or less. The oligonucleotides may be single stranded or double stranded.

The term “complementary” or “complementarity” means that two nucleic acid sequences are capable of at least partially base-pairing according to the standard Watson-Crick complementarity rules. For example, two sticky ends can be partially complementary, wherein a region of one overhang complements and anneals with a region or all of another overhang. The gap(s) can be filled in by chain extension in the presence of a polymerase and single nucleotides, followed by or simultaneously with a ligation reaction.

A “construct” refers to a DNA sequence which includes a complete target sequence. Generally it is implied that the construct has been assembled. A “subconstruct” means a portion of the complete target sequence that typically is an intermediate product during hierarchical assembly.

As used herein, an “eluate” refers to a mixture of a plurality of oligonucleotides, cleaved off or otherwise removed from a chip and pooled into a solution.

“Library” used herein refers to a diverse collection or mixture of oligonucleotides (e.g., terminators). In certain embodiments, a library may comprise at least 2, 3, 4, 5, 6, 7, 8, 9, 10 . . . 2⁴, 3⁴, 4⁴, 5⁴ . . . N⁴ members wherein N is any integer (e.g., representing the length of the degenerate portion in the terminator).

“Nucleic acid,” “nucleic acid sequence,” “oligonucleotide,” “polynucleotide,” “gene” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. As used herein, an “oligonucleotide” may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 300 nucleotides long (e.g., from about 30 to 250, from about 40 to 220 nucleotides long, from about 50 to 200 nucleotides long, from about 60 to 180 nucleotides long, or from about 65 or about 150 nucleotides long), between about 100 and about 200 nucleotides long, between about 200 and about 300 nucleotides long, between about 300 and about 400 nucleotides long, or between about 400 and about 500 nucleotides long. However, shorter or longer oligonucleotides may be used. An oligonucleotide may be a single-stranded or double-stranded nucleic acid. As used herein the terms “nucleic acid”, “polynucleotide” and “oligonucleotide” are used interchangeably and refer to naturally-occurring or non-naturally occurring, synthetic polymeric forms of nucleotides. In general, the term “nucleic acid” includes both “polynucleotide” and “oligonucleotide” where “polynucleotide” may refer to longer nucleic acid (e.g., more than 1,000 nucleotides, more than 5,000 nucleotides, more than 10,000 nucleotides, etc.) and “oligonucleotide” may refer to shorter nucleic acid (e.g., 10-500 nucleotides, 20-400 nucleotides, 40-200 nucleotides, 50-100 nucleotides, etc.).

The nucleic acid molecules of the present disclosure may be formed from naturally occurring nucleotides, for example forming deoxyribonucleic acid (DNA) or ribonucleic acid (RNA) molecules. Alternatively, naturally-occurring nucleic acids may include structural modifications to alter their properties, such as in peptide nucleic acids (PNA) or in locked nucleic acids (LNA). The solid phase synthesis of nucleic acid molecules with naturally occurring or artificial bases is well known in the art. The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. In some embodiments, the sequence of the nucleic acids does not exist in nature (e.g., a cDNA or complementary DNA sequence, or an artificially designed sequence).

Usually in a nucleic acid nucleosides are linked by phosphodiester bonds. Whenever a nucleic acid is represented by a sequence of letters, it will be understood that the nucleosides are in the 5′ to 3′ order from left to right. In accordance to the IUPAC notation, “A” denotes deoxyadenosine, “C” denotes deoxycytidine, “G” denotes deoxyguanosine, “T” denotes deoxythymidine, “U” denotes the ribonucleoside, uridine. In addition, there are also letters which are used when more than one kind of nucleotide could occur at that position: “W” (i.e. weak bonds) represents A or T, “S” (strong bonds) represents G or C, “M” (for amino) represents A or C, “K” (for keto) represents G or T, “R” (for purine) represents A or G, “Y” (for pyrimidine) represents C or T, “B” represents C, G or T, “D” represents A, G or T, “H” represents A, C or T, “V” represents A, C, or G and “N” represents any base A, C, G or T (U). It is understood that nucleic acid sequences are not limited to the four natural deoxynucleotides but can also comprise ribonucleoside and non-natural nucleotides. A “/” in a nucleotide sequence or nucleotides given in brackets refer to alternative nucleotides, such as alternative U in a RNA sequence instead of T in a DNA sequence. Thus, U/T or U(T) indicate one nucleotide position that can either be U or T. Likewise, A/T refers to nucleotides A or T; G/C refers to nucleotides G or C. Due to the functional identity between U and T any reference to U or T herein shall also be seen as a disclosure as the other one of T or U. For example, the reference to the sequence UUCG (on an RNA) shall also be understood as a disclosure of the sequence TTCG (on a corresponding DNA). For simplicity only, only one of these options is described herein. Complementary nucleotides or bases are those capable of base pairing such as A and T (or U); G and C; G and U.

“Parse” when used as a verb refers to the process of breaking a target sequence into compatible fragments for assembly. In some embodiments, the compatible fragments have compatible sticky ends that enable the fragments to be ligated in a predetermined order. When used as a noun a “parse” means a set of fragments which may be assembled together into a larger DNA sequence.

In general, a “stem-loop” sequence (used interchangeably with “hairpin”) refers to a sequence in which at least two regions within a single DNA or RNA molecule that are reverse compliments of each other are separated by one or more non-complimentary region, such that the complementary regions hybridize and form a “stem,” while the non-complementary region forms a “loop.”

A “target” means a nucleic acid of a known nucleotide sequence (e.g., as ordered by a customer) to be synthesized or assembled using one or more methods disclosed herein.

A “terminator”, “terminator oligonucleotide” or “terminator oligo” refers to a nucleic acid sequence comprising a stem-loop in which the free end of the stem portion can be attached (e.g., ligated) to another nucleic acid sequence (e.g., a construction oligonucleotide or a subconstruct). The stem portion can be designed to have a high melting temperature (e.g., higher than 65° C., higher than 68° C., higher than 70° C. or higher than 72° C.). Once attached, the stem portion tends to self-anneal when the temperature is lower than its melting temperature, thereby preventing the nucleic acid it is attached to from participating in further reaction such as polymerase chain reaction. The terminator can also be designed to include one or more labels (e.g., biotin and digoxigenin) to facilitate removal of the terminator (along with the nucleic acid it is attached to) upon binding to its binding partner.

As used herein, “including,” “comprising,” “having,” “containing,” “involving,” and variations thereof, are meant to encompass the items listed thereafter and equivalents thereof as well as additional items. “Consisting of” shall be understood as a close-ended relating to a limited range of elements or features. “Consisting essentially of” limits the scope to the specified elements or steps but does not exclude those that do not materially affect the basic and novel characteristics of the claimed invention.

Other terms used in the fields of recombinant nucleic acid technology, synthetic biology, and molecular biology as used herein will be generally understood by one of ordinary skill in the applicable arts.

### Synthetic Oligonucleotides

Typically, oligonucleotide synthesis involves a number of chemical steps that are performed in a cycle repetitive manner throughout the synthesis with each cycle adding one nucleotide to the growing oligonucleotide chain. The chemical steps involved in a cycle are a deprotection step that liberates a functional group for further chain elongation, a coupling step that incorporates a nucleotide into the oligonucleotide to be synthesized, and other steps as required by the particular chemistry used in the oligonucleotide synthesis, such as an oxidation step required with the phosphoramidite chemistry. Optionally, a capping step that blocks those functional groups which were not elongated in the coupling step can be inserted in the cycle. The nucleotide can be added to the 5′-hydroxyl group of the terminal nucleotide, in the case in which the oligonucleotide synthesis is conducted in a 3′→5′ direction or at the 3′-hydroxyl group of the terminal nucleotide in the case in which the oligonucleotide synthesis is conducted in a 5′→3′ direction.

For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments the P strand may be a sense strand of a coding sequence, whereas in other embodiments the P strand may be an anti-sense strand of a coding sequence. It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA.

In some aspects of the disclosure, the oligonucleotides synthesized or otherwise prepared according to the methods described herein can be used as building blocks for the assembly of a target polynucleotide of interest.

Oligonucleotides may be synthesized on solid support. As used herein, the term “solid support”, “support” and “substrate” are used interchangeably and refers to a porous or non-porous solvent insoluble material on which polymers such as nucleic acids are synthesized or immobilized. As used herein “porous” means that the material contains pores having substantially uniform diameters (for example in the nm range). Porous materials can include but are not limited to, paper, synthetic filters and the like. In such porous materials, the reaction may take place within the pores. The support can have any one of a number of shapes, such as pin, strip, plate, disk, rod, bends, cylindrical structure, particle, including bead, nanoparticle and the like. In some embodiments, the support is planar (e.g., a chip). The support can have variable widths.

The support can be hydrophilic or capable of being rendered hydrophilic. The support can include inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, ceramics, metals, and the like; either used by themselves or in conjunction with other materials.

In some embodiments, pluralities of different single-stranded oligonucleotides are immobilized at different features of a solid support. In some embodiments, the support-bound oligonucleotides may be attached through their 5′ end or their 3′ end. In some embodiments, the support-bound oligonucleotides may be immobilized on the support via a nucleotide sequence (e.g., degenerate binding sequence), linker (e.g., photocleavable linker or chemical linker). It should be appreciated that by 3′ end, it is meant the sequence downstream to the 5′ end and by 5′ end it is meant the sequence upstream to the 3′ end. For example, an oligonucleotide may be immobilized on the support via a nucleotide sequence or linker that is not involved in subsequent reactions.

Certain embodiments of the disclosure may make use of a solid support comprised of an inert substrate and a porous reaction layer. The porous reaction layer can provide a chemical functionality for the immobilization of pre-synthesized oligonucleotides or for the synthesis of oligonucleotides. In some embodiments, the surface of the array can be treated or coated with a material comprising suitable reactive group for the immobilization or covalent attachment of nucleic acids. Any material, known in the art, having suitable reactive groups for the immobilization or in situ synthesis of oligonucleotides can be used.

In some embodiments, the porous reaction layer can be treated so as to comprise hydroxyl reactive groups. For example, the porous reaction layer can comprise sucrose.

According to some aspects of the disclosure, oligonucleotides terminated with a 3′ phosphoryl group oligonucleotides can be synthesized a 3′→5′ direction on a solid support having a chemical phosphorylation reagent attached to the solid support. In some embodiments, the phosphorylation reagent can be coupled to the porous layer before synthesis of the oligonucleotides. In an exemplary embodiment, the phosphorylation reagent can be coupled to the sucrose. For example, the phosphorylation reagent can be 2-[2-(4,4′-Dimethoxytrityloxy)ethylsulfonyl]ethyl-(2-cyanoethyl)-(N,N-diisopropyl)-phosphoramidite. In some embodiments, the 3′ phosphorylated oligonucleotide can be released from the solid support and undergo subsequent modifications according to the methods described herein. In some embodiments, the 3′ phosphorylated oligonucleotide can be released from the solid support using ammonium hydroxide.

In some embodiments, synthetic oligonucleotides for the assembly may be designed (e.g., sequence, size, and number). Synthetic oligonucleotides can be generated using standard DNA synthesis chemistry (e.g., phosphoramidite method). Synthetic oligonucleotides may be synthesized on a solid support, such as a microarray, using any appropriate technique as described in more detail herein. Oligonucleotides can be eluted from the microarray prior to be subjected to amplification or can be amplified on the microarray. It should be appreciated that different oligonucleotides may be designed to have different lengths.

In some embodiments, oligonucleotides are synthesized (e.g., on an array format) as described in U.S. Pat. No. 7,563,600, U.S. patent application Ser. No. 13/592,827, and WO 2014/004393, which are hereby incorporated by reference in their entireties. For example, single-stranded oligonucleotides are synthesized in situ on a common support wherein each oligonucleotide is synthesized on a separate or discrete feature (or spot) on the substrate. In some embodiments, single-stranded oligonucleotides are bound to the surface of the support or feature. As used herein, the term “array” refers to an arrangement of discrete features for storing, routing, amplifying and releasing oligonucleotides or complementary oligonucleotides for further reactions. The array can be planar. In an embodiment, the support or array is addressable: the support includes two or more discrete addressable features at a particular predetermined location (i.e., an “address”) on the support. Therefore, each oligonucleotide molecule of the array is localized to a known and defined location on the support. The sequence of each oligonucleotide can be determined from its position on the support. Moreover, addressable supports or arrays enable the direct control of individual isolated volumes such as droplets. The size of the defined feature can be chosen to allow formation of a microvolume droplet on the feature, each droplet being kept separate from each other. As described herein, features are typically, but need not be, separated by interfeature spaces to ensure that droplets between two adjacent features do not merge. Interfeatures will typically not carry any oligonucleotide on their surface and will correspond to inert space. In some embodiments, features and interfeatures may differ in their hydrophilicity or hydrophobicity properties.

An oligonucleotide may be a single-stranded nucleic acid. However, in some embodiments a double-stranded oligonucleotide may be used as described herein. In certain embodiments, an oligonucleotide may be chemically synthesized as described herein. In some embodiments, nucleic acids (e.g., synthetic oligonucleotide) may be amplified before use. The resulting product may be double-stranded. One or more modified bases (e.g., a nucleotide analog) can be incorporated. Examples of modifications include, but are not limited to, one or more of the following: methylated bases such as cytosine and guanine; universal bases such as nitro indoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH2 (deoxyribose-NEb); Acridine (6-chloro-2-methoxiacridine); and spacer phosphoramides which are used during synthesis to add a spacer “arm” into the sequence, such as C3, C8 (octanediol), C9, C12, HEG (hexaethlene glycol) and C18.

In various embodiments, the synthetic single-stranded or double-stranded oligonucleotides can be non-naturally occurring, e.g., being unmethylated or modified in a way (e.g., chemically or biochemically modified in vitro) such that they become hemi-methylated (only one strand is methylated) or semi-methylated (only a portion of the normal methylation sites are methylated on one or both strands) or hypomethylated (more than the normal methylation sites are methylated on one or both strands), or have non-naturally occurring methylation patterns (some of the normal methylation sites are methylated on one or both strands and/or normally unmethylated sites are methylated). In contrast, naturally-occurring DNA typically contains epigenetic modifications such as methylation at, e.g., the C-5 position of the cytosine ring of DNA by DNA methyltransferases (DNMTs) in vivo. DNA methylation is reviewed by Jin et al., Genes & Cancer 2011 June; 2(6): 607-617, which is incorporated herein by reference in its entirety.

### Design of Terminator Oligonucleotides

In one aspect, non-naturally occurring, artificial terminators are provided herein. In some embodiments, the terminator can include one or more stem-loop sequences. In some cases, a stem-loop sequence can be about 7 to about 200 nucleotides in length, between 10 and 100 nucleotides in length, between 15 and 80 nucleotides in length, between 20 and 50 nucleotides in length, or between 25 and 40 nucleotides in length. The stem-loop sequence may be shorter or longer depending on the design.

Within each stem-loop, one or more loop structures can be designed. The loop can be a full loop where the two nucleotides at the base of the loop and connecting with the stem are complementary (e.g., A-T or G-C). Generally the loop at the top of the stem is a full loop. The loop can also be a half loop if the two nucleotides at the base of the loop and connecting with the stem do not form a base pair (e.g., A and A, T and T, A and G, T and C, etc.). A stem-loop can have one or more full loops and/or half loops. The size of the loop, excluding the two nucleotides at the base of the loop and connecting with the stem, can be anywhere between 3-12 nucleotides, or between 4-10 nucleotides, or between 5-8 nucleotides, if the host is bacterium such as E. coli. If the host is yeast or a mammalian cell, the loop size can be larger, e.g., up to 15 nucleotides or up to 20 nucleotides or larger.

The stem portion does not need to have 100% complementarity between the two base-paring fragments. For convenience, one fragment in the stem is name positive or + fragment while the other negative or − fragment. In some embodiments, the stem can have at least about 98%, at least about 95%, at least about 90%, at least about 85%, at least about 80%, at least about 75%, at least about 70%, at least about 60%, or at least about 50% of complementarily between the two base-paring fragments. Where there is less than 100% complementarily, the positive fragment may contain, compared to the negative fragment, one or more mismatches, one or more insertions (consecutively so as to form a loop or non-consecutively) and/or one or more deletions (consecutively so as to form a loop on the negative fragment or non-consecutively).

The stem portion can be designed to have a high melting temperature (e.g., higher than 65° C., higher than 68° C., higher than 70° C. or higher than 72° C.). As such, the stem portion tends to self-anneal when the temperature is lower than its melting temperature, thereby forming a hairpin structure. Once attached to another nucleic acid, the hairpin can act to prevent the nucleic acid it is attached to from participating in further reaction such as polymerase chain reaction. To that end, the stem portion can be designed to have a high GC content, e.g., higher than 50%, higher than 60%, or higher than 70%.

In certain embodiments, the free end of the stem portion can be attached (e.g., ligated) to another nucleic acid sequence (e.g., a construction oligonucleotide or a subconstruct). The free end of the stem portion can be blunt ended or contain a sticky end. The sticky end may be a 5′ or 3′ overhang. The overhang can be any length, e.g., 1-100 nucleotides, 1-50 nucleotides, 1-20 nucleotides, or 2-10 nucleotides. In some embodiments, the overhang is 4-8, 4-6, or 4 nucleotides long. The overhang can contain a degenerate sequence to enable annealing and ligation with essentially any sequence. For example, the overhang can be a 4-nucleotide long degenerate sequence and have 4̂4=256 possible sequences. In some embodiments, a library of terminators can be provided where all possible sequences of the degenerate overhang portion are included, with a universal stem-loop sequence or more than one stem-loop sequences.

The terminator can also be designed to include one or more labels to facilitate removal of the terminator (and/or the nucleic acid it is attached to) upon binding to its binding partner. The label can be biotin and/or digoxigenin. Terminators can be labeled according to methods known in the art. The labels can be enzymatically introduced into the terminator via labeled nucleotides either directly (e.g., using biotinylated/digoxigenylated nucleotides) or indirectly via incorporation of a nucleotide analog carrying a reactive group and subsequent biotinylation/digoxigenylation. Oligonucleotides can also be biotinylated according to the methods described in York et al. (Nucleic Acids Research, 2012, Vol. 40, No. 1 e4), which is incorporated herein by reference in its entirety.

The labeled terminators (and molecules to which they are attached) can be affinity purified and/or removed. As one of ordinary skill in the art would understand, binding partners for biotin include avidin, streptavidin and NeutrAvidin. Digoxigenin (DIG) binding partners include anti-DIG antibodies. The binding partners can be conjugated to a capture means such as beads, a column or any other solid surface, to facilitate the subsequent removal of the labeled terminators and molecules to which they are attached.

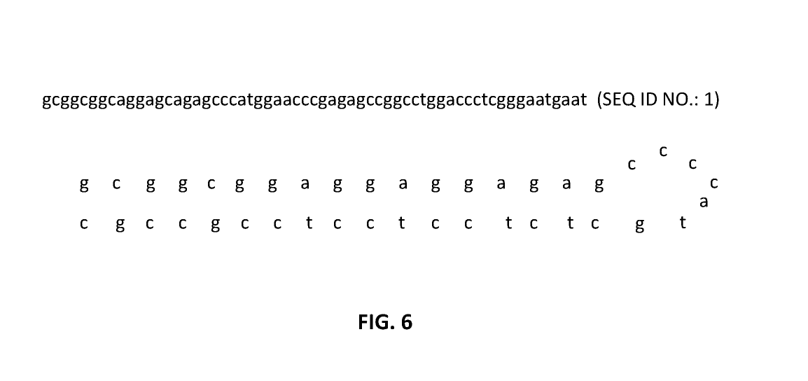

In some embodiments, the present disclosure provides a non-naturally occurring nucleic acid sequence comprising a Y—X—Z—O stem-loop, wherein: Y is a nucleotide sequence of 5 to 30 nucleotides in length; X is a nucleotide sequence of 3 to 12 nucleotides in length, each nucleotide therein not base pairing with any other nucleotide within X; Z is a nucleotide sequence of 5 to 50 nucleotides in length and having at least 70% complementarity to Y; and O is either absent or an overhang protruding from a stem formed between Y and Z. X is the loop portion of the stem-loop and may be 3-8 nucleotides in length, 4-6 nucleotides in length or 5-6 nucleotides in length in some embodiments. The stem-loop in some embodiments can include one or more dT-biotin. In some examples, the stem-loop can have the sequence of SEQ ID NO.: 1 as shown in FIG. 6.

In some embodiments, Y has a G/C content of at least 60%, at least 50%, or at least 40%. Y may be 5′ to X or 3′ to X. Y can be, in certain embodiments, 12-18 nucleotides in length, 14-16 nucleotides in length, 16-18 nucleotides in length, 17-19 nucleotides in length, 15-30 nucleotides in length, 18-27 nucleotides in length, 21-24 nucleotides in length, 24-28 nucleotides in length, or 25-29 nucleotides in length. In some embodiments, Y is of 10, 11, 12, 13, 14, 15, 16, 17, 18, 19, 20, 21, 22, 23, 24, 25, 26, 27, 28, 29, or 30 nucleotides in length.

The length of Y determines the length of Z (by complementarity), which can be selected to have substantially the same nucleotide length as Y. Z can have the same length as Y and may have one or more mismatches with Y. Z can also have one or more insertions compared to Y, thereby forming one or more protrusions or loops when annealed with Y. The length of substantially complementary Y and Z, the stem of the hairpin, determines the stem length in base pairs. The stem is not necessarily 100% complementary as described herein, but can have limited non-complementary opposing bases for Y and Z.

In particular, Y and Z can be of m and n nucleotides in length, respectively, where Y consists of the nucleotides y₁ to yₘ and Z consists of the nucleotides z₁ to zₙ. Preferably z₁ is complementary to y₁ and zₙ is complementary to yₘ so that the end points of the stem of the hairpin are complementary. Y and Z can be at least 60% complementary, preferably at least 70%, at least 80%, at least 82%, at least 84%, at least 85%, at least 86%, at least 88%, at least 90%, at least 92%, at least 94%, at least 95%, at least 96%, at least 97%, at least 98%, at least 99%, or even 100%, complementary. The complementarity is most preferably at least 70%, preferably at least 75%, at least 80%, at least 85%, at least 90%, at least 95% or at least 100. Non-complementarities such as mismatches, insertions and/or deletions are possible but should be limited. Some limited non-complementarities may be placed adjacent to each other to form one or more additional loops.

O may be absent in the case of a blunt-ended terminator. O can also be a 5′ or 3′ overhang when the terminator has a sticky end. O can be 1-100 nucleotides, 1-50 nucleotides, 1-20 nucleotides, 2-10 nucleotides, 4-8 nucleotides, 4-6 nucleotides, or 4 nucleotides long. O can include a degenerate sequence.

The terminator can also be designed to include one or more restriction enzyme (RE) sites. The RE sites can be any type II RE sites such as type IIP or IIS and modified or hybrid sites. Type IIP enzymes recognize symmetric (or palindromic) DNA sequences 4 to 8 base pairs in length and generally cleave within that sequence. Examples: EcoRI, HindIII, BamHI, NotI, PacI, MspI, HinP1I, BstNI, NciI, SfiI, NgoMIV, EcoRI, HinfI, Cac8I, AlwNI, PshAI, BglI, XcmI, HindIII, NdeI, SacI, PvuI, EcoRV, NciI, TseI, PspGI, BglII, ApoI, AccI, BstNI, and NciI. Type IIS restriction enzymes make a single double stranded cut 0-20 bases away from the recognition site. Examples include but are not limited to BstF5I, BtsCI, BsrDI, BtsI, AlwI, BccI, BsmAI, EarI, MlyI (blunt), PleI, BmrI, BsaI, BsmBI, FauI, MnlI, SapI, BbsI, BciVI, HphI, MboII, BfuAI, BspCNI, BspMI, SfaNI, HgaI, BseRT, BbvI, EciI, FokI, BceAI, BsmFI, BtgZI, BpuEI, BsgI, MmeI, BseGI, Bse3DI, BseMI, AcIWI, Alw26I, Bst6I, BstMAI, Eam1104I, Ksp632I, PpsI, SchI (blunt), BfiI, Bso31I, BspTNI, Eco31I, Esp3I, SmuI, BfuI, BpiI, BpuAI, BstV2I, AsuHPI, Acc36I, LweI, AarI, BseMII, TspDTI, TspGWI, BseXI, BstVlI, Eco57I, Eco57MI, GsuI, and BcgI. Such enzymes and information regarding their recognition and cleavage sites are available from commercial suppliers such as New England Biolabs, Inc. (Ipswich, Mass., U.S.A.).

The RE sites can be methylated such that they can be digested with a methylation-sensitive nuclease such as MspJI, SgeI and FspEI. Such nuclease shares both type IIM and type IIS properties; thus, it only recognizes the methylation-specific 4-bp sites, ^{m}CNNR (N=A or T or C or G; R=A or G), and cuts DNA outside of this recognition sequences.

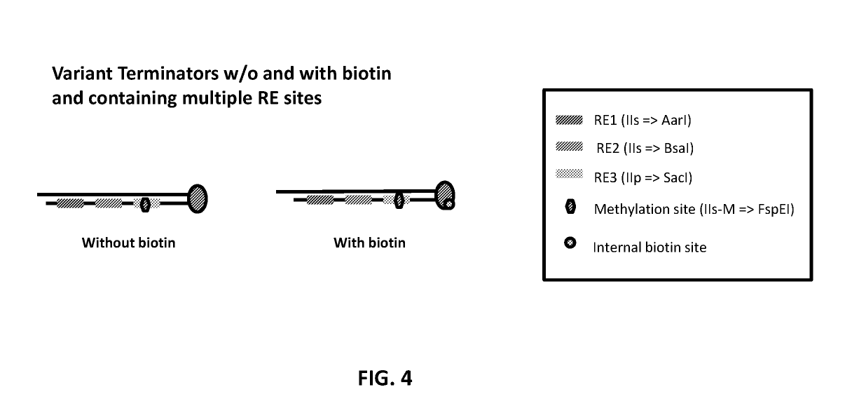

As shown in FIG. 4, the terminator can include more than one RE sites, e.g., 2, 3 or more RE sites. One or more RE sites can be type IIS sites such as AarI and BsaI sites. One or more RE sites can be type IIP sites such as SacI. One or more RE sites can be recognized by hybrid type IIS-M RE such as FspEI.

The terminator or library of terminators can be chemically synthesized. In some embodiments, the terminators can be synthesized on a chip which can be the same chip that carries the construction oligonucleotides, or a different chip. After synthesis, the terminators can be cleaved and eluted off of the chip into the same eluate, or a different eluate, as the construction oligonucleotides.

In some embodiments, the terminator may be biotinylated in the stem portion and/or the loop portion (e.g., via dT-biotin). Terminators can be biotinylated according to methods known in the art. For example, oligonucleotides can be biotinylated according to the methods described in York et al. (Nucleic Acids Research, 2012, Vol. 40, No. 1 e4), which is incorporated herein by reference in its entirety. The biotinylated products can be affinity purified using avidin, streptavidin or NeutrAvidin (which can be, e.g., bound to a bead, column, or other surface).

### Use of Terminator Oligonucleotides in Assembly

In some embodiments, the terminator oligos disclosed herein can be used in one or more assembly steps. For example, during subassembly and/or assembly, incomplete subassembly or assembly products (e.g., where one or more construction oligonucleotides are missing from the subconstruct or final construct) may be present. These incomplete products are difficult to remove or separate from the completely and correctly assembled products due to proximity in size. In subsequence polymerase based amplification, the incomplete molecules are often amplified along with the correct assembly products. To suppress amplification of the incomplete products, terminator oligonucleotides can be attached to one or both ends of the subassembly or assembly products, such that amplification of the incomplete products is disfavored without affecting amplification of correct assembly products.

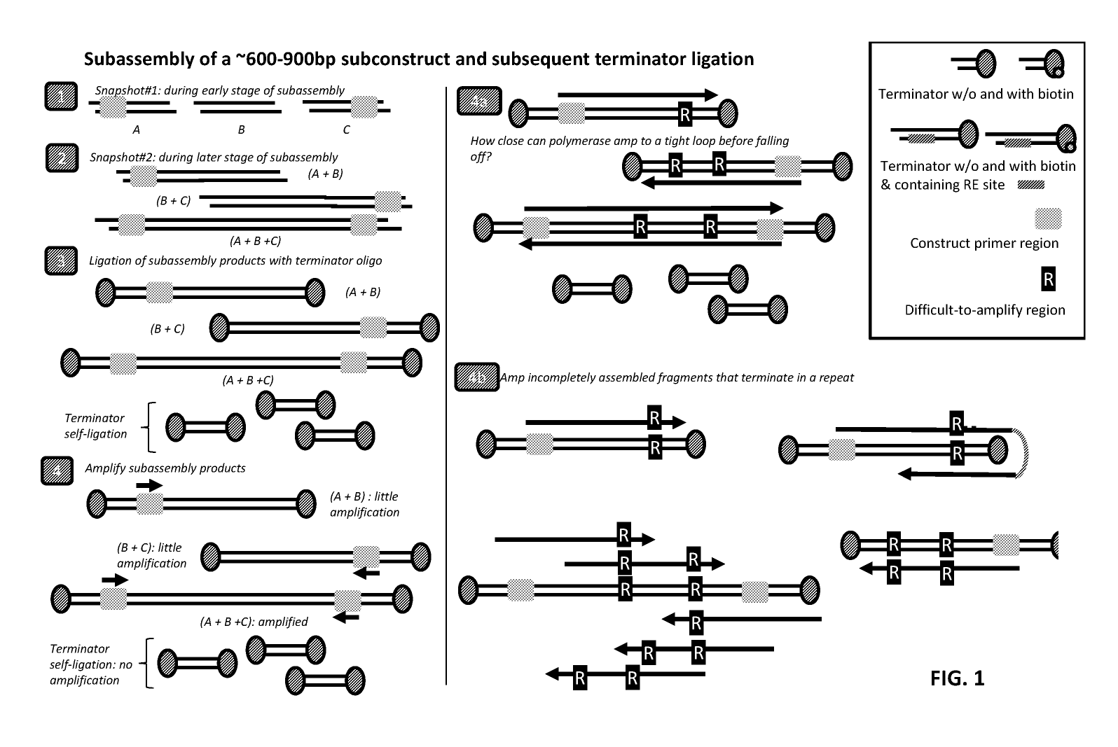

FIG. 1 illustrates an exemplary use of terminator oligonucleotides during subassembly of subconstructs (e.g., 600-900 nucleotides long). In step 1, during early stage of subassembly, construction oligonucleotides A, B and C are present in a solution, e.g., an eluate from a chip on which the construction oligonucleotides were synthesized. Construction oligonucleotides A, B and C can also be amplification products of the direct eluate from the chip, using (1) one or more universal primers against universal primer biding site(s) therein, or (2) one or more specific primers against individual primer biding site(s) therein, or (3) both (1) and (2). To the extent the universal primer biding site(s) are extraneous and are not part of the target nucleic acid to be assembled, restriction enzyme digestion can be used to both remove the extraneous sequences and produce desirable cohesive ends. The identity of the cohesive ends determines the particular order of assembly, i.e., each pair of cohesive ends are unique enough to ensure the 3′ end of A only anneals and ligates with the 5′ end of B, and the 3′ end of B only anneals and ligates with the 5′ end of C.

Referring to FIG. 1, step 1, A and C can each be designed to have a primer region for later amplification of the subassembly product (step 4), as well as a sticky end for later ligation with terminator oligos (step 3). The primer region can be universal or specific. The primer region can be designed to be part of the construction oligonucleotides that are incorporated into the final target nucleic acid. In some embodiments, all or part of each primer region can be in the form of a flanking region outside the central portion of a construction oligonucleotide, wherein the central portion is incorporated into the final target nucleic acid and the flanking region needs be removed before assembly. To that end, one or more restriction sites can be designed to allow removal of the flanking region.

In step 2 of FIG. 1, during later stage of subassembly, complete assembly product (A+B+C) as well as incomplete assembly products (A+B) and (B+C) are present.

In step 3 of FIG. 1, subassembly products can be ligated with terminator oligos. In addition, terminator oligos may self-ligate to form double hairpins. The terminator oligos can each have an overhang of a degenerate sequence (e.g., 4 nucleotides long), to enable annealing and ligation with any other overhangs.

In step 4 of FIG. 1, ligated subassembly products are amplified using primers specific for the primer region that is predesigned in construction oligonucleotides A and C as described above. Without wishing to be bound by theory, it is believed that due to one or more of the following reasons, amplification of the incomplete assembly products is reduced or suppressed: (1) at PCR extension temperature (e.g., 72° C. or other temperature depending on the polymerase used and manufacturer's recommendation), the high melting temperature of the stem portion in the terminator allows the terminator to stay in its stem-loop structure, which prevents the polymerase from further proceeding; and (2) the terminator physically links the two complementary strands of a double-stranded nucleic acid together and makes them more likely to anneal with each other (due to intramolecular reaction) as opposed to with primers (intermolecular reaction).

Terminators are particularly useful in situations where the target nucleic acid has a region that is difficult to amplify or assemble due to, e.g., high GC content, low GC content, secondary structure, and/or repeat sequences. As illustrated in FIG. 1, steps 4a and 4b, the complete assembly products can be amplified.

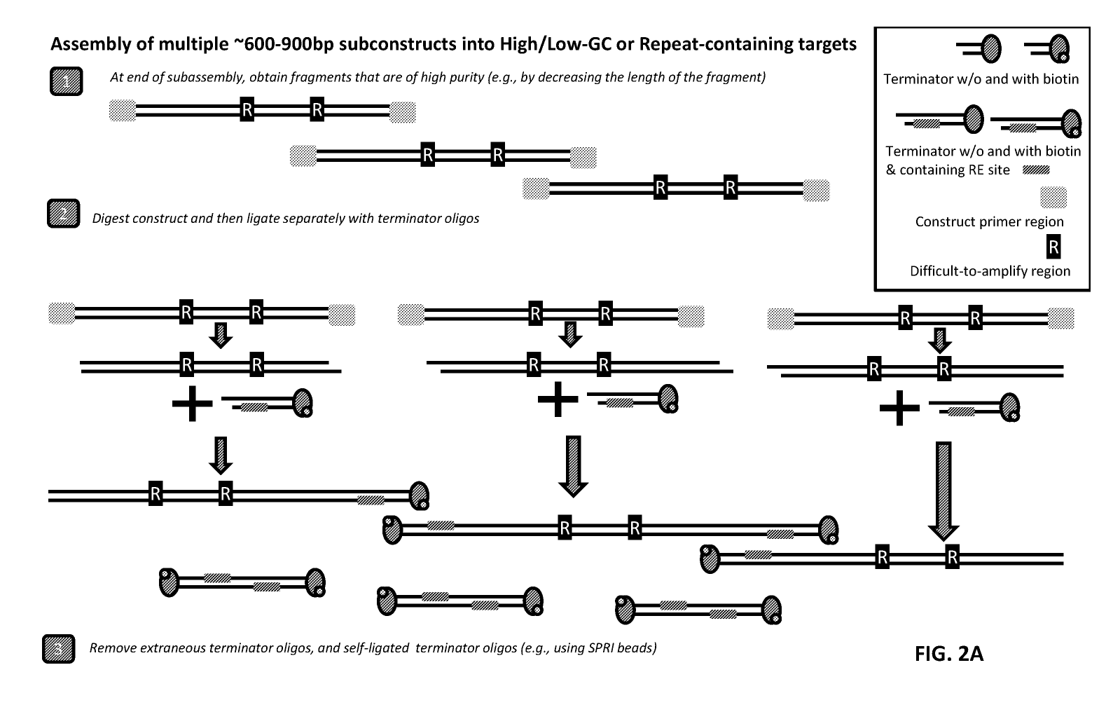

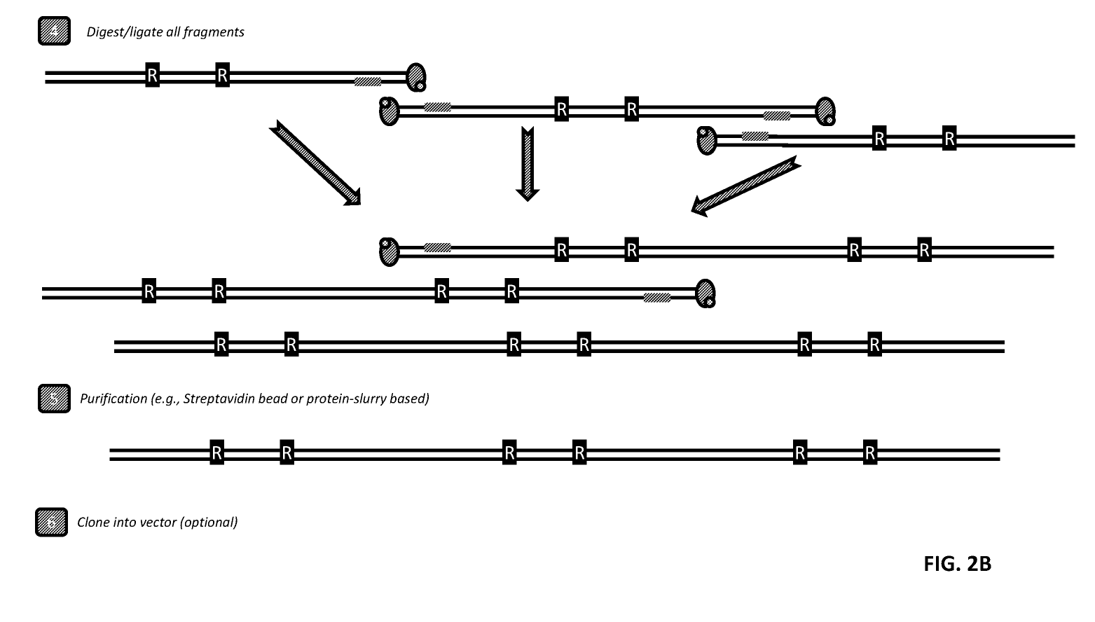

After subassembly, two or more subconstructs can be assembled into the final target. FIGS. 2A-2B illustrate an exemplary use of terminator oligonucleotides during assembly of target. The terminator oligos in this example contain an RE site and a label. It should be noted that while biotin is used as an example for illustration purpose only, other labels such a DIG can also be used. In addition, more than one RE sites can be engineered in the terminators.

In step 1, FIG. 2A, at the end of subassembly, obtain subconstructs that are of high purity, which can be achieved by, e.g., decreasing the length of the subconstructs. Each subconstruct can have universal or unique primer regions at both ends. The primer regions can be removed by restriction enzyme digestion to expose desirable blunt or sticky ends. For example, as shown in step 2 of FIG. 2A, the two terminal subconstructs can be designed such that upon digestion, they each have one blunt end and one sticky end, whereas the central subconstruct has two sticky ends. This way, upon ligation with terminators, the two terminal subconstructs each has one terminator attached whereas the central construct has two terminators attached at both ends, as shown in step 2. Self-ligated terminators are also present. Next in step 3, extraneous terminators and self-ligated terminators can be removed by size selection. For example, Solid Phase Reversible Immobilization (SPRI) beads from Beckman Coulter can be used. In step 4, FIG. 2B, the terminator-attached subconstructs can be subject to digestion via, e.g., restriction enzyme sites that are previously engineered in the terminator oligonucleotides, and ligation of compatible sticky ends. The digestion and ligation reactions can take place in a one-pot reaction. The result is both partially or incompletely assembled products that still have terminators attached thereto (e.g., due to incomplete digestion), as well as terminator free, completely assembled target. The incomplete assembly products can be removed using streptavidin beads or protein-slurry based purification. The purified complete assembly product can then be further amplified, sequenced and/or cloned into a vector.

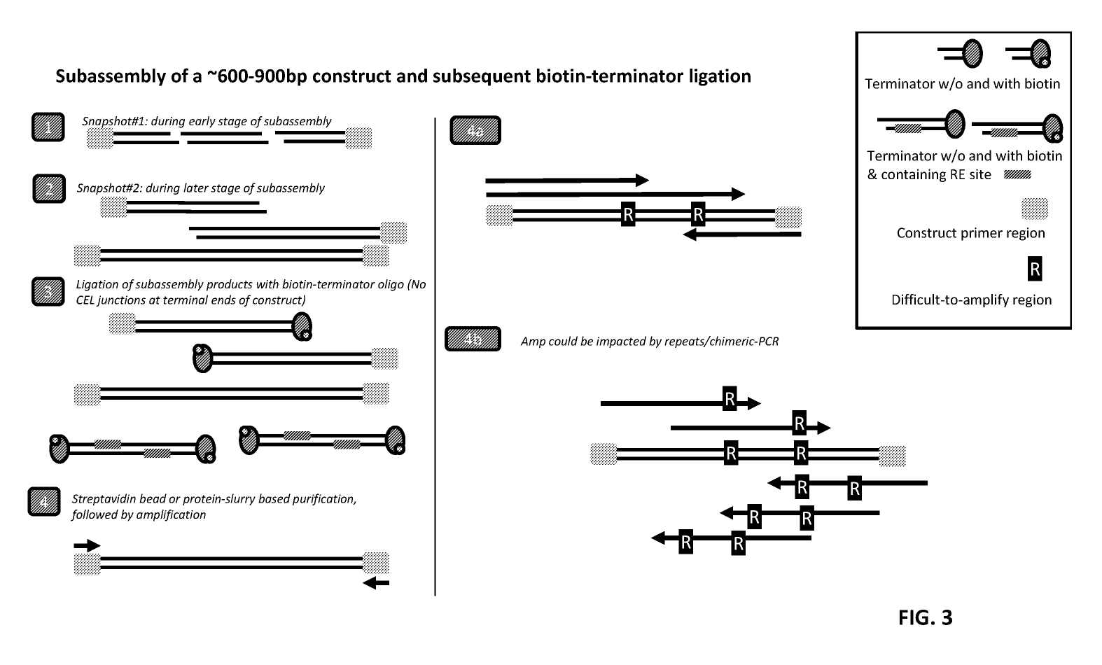

Another assembly strategy uses biotin-labeled terminators to facilitate subassembly. FIG. 3 illustrates an exemplary use of biotin-terminator oligonucleotides during subassembly. Steps 1-3 are similar to FIG. 1, except that the terminal construction oligonucleotides each have a blunt end that does not ligate with terminators. As a result, the complete subassembly products do not ligate with terminators while incomplete subassembly products do. In step 4, the complete subassembly product is purified using streptavidin beads or protein-slurry based purification, followed by amplification.

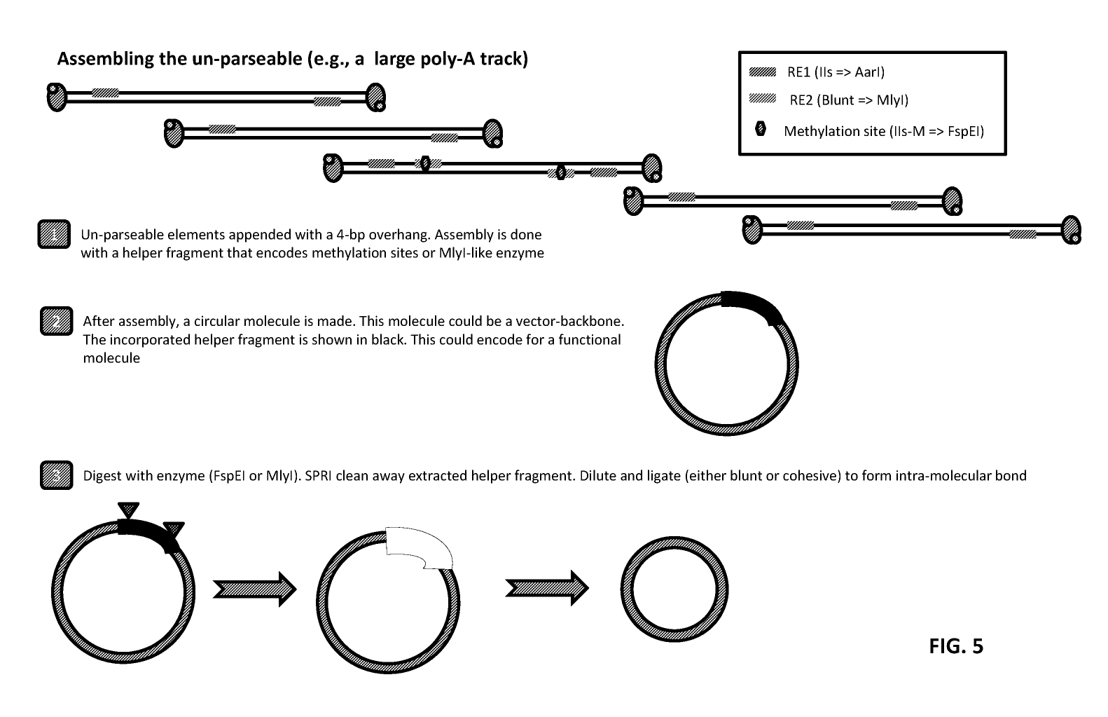

FIG. 5 illustrates a further exemplary assembly strategy. This is particularly useful in cases where the target or a portion thereof is un-parseable, i.e., unable to parse the sequence into compatible fragments for assembly. One example is a large poly-A track (A)n or other repeat sequences where it is impossible to ensure that n number of A's are assembled. As shown in FIG. 5, step 1, un-parseable elements can be appended with an overhang (e.g., 4 nucleotides long) and assembled with a helper fragment that contains one or more methylated RE sites (e.g., FspEI site) or sites recognized by blunt-end generating enzyme such as MlyI. After assembly as shown in step 2, a circular molecule is produced. The circular molecule can be a vector backbone. In step 3, the circular molecule is digested to remove the helper fragment using, e.g., FspEI or MlyI. The helper fragment is cleaned away using, e.g., SPRI beads. The remaining digestion product can be ligated to form intramolecular bond.

The target polynucleotide can be produced in a one-pot reaction where all construction oligonucleotides are mixed and ligated together. Ligation can also be performed sequentially (ligating oligonucleotides one by one) or hierarchically (ligating subpools of the oligonucleotides into one or more subconstructs which are then ligated into the final target construct). It should be noted that one or more of the construction oligonucleotides, one or more of the subconstructs, and/or the final target construct can be non-naturally occurring, e.g., being unmethylated or modified in a way (e.g., chemically or biochemically modified in vitro) such that they become hemi-methylated or semi-methylated or hypomethylated, or have non-naturally occurring methylation patterns. Such non-naturally occurring methylation and methylation patterns can be used to regulate, for example, gene expression.

Amplification of the construction oligonucleotides before assembly is optional. In some embodiments, all of the construction oligonucleotides can be assembled together without amplification (e.g., when the construction oligonucleotides are provided at relatively uniform and sufficient amount). In certain embodiments, only a subset of the construction oligonucleotides (e.g., those that are underrepresented) are amplified before assembly. Assembly can be done in one step, or hierarchically in more than one step, or sequentially by adding one construction oligonucleotide at a time, or any combination of the foregoing where some construction oligonucleotides are assembled using one method while others are assembled using another method. In one example, the construction oligonucleotides may, without amplification, be first assembled into two or more subconstructs. The subconstructs can be optionally amplified and then assembled, in one or more steps, into the final construct having the predetermined target sequence.

Methods and compositions of the present disclosure can be used in the assembly of long-length polynucleotides (e.g., 10 kb or longer). In certain embodiments, small oligonucleotides (e.g., 100-800 bp or 500-800 bp) synthesized off of a chip can be first assembled into an intermediate polynucleotide, with or without using methods and compositions of the present disclosure. The intermediate polynucleotide can then be cloned into a plasmid, which can be introduced into a host, amplified via culturing, isolated and purified, cleaved using methods and compositions of the present disclosure, and then subjected to further assembly. This process can be repeated multiple times till the final long-length product is assembled.

In addition or as an alternative to direct ligation or polymerase assisted assembly, other methods can also be used to assemble cleavage products of the present disclosure. In some embodiments, the cleavage products can be subject to homologous recombination via SLiCE (Seamless Ligation Cloning Extract), as described in, for example, Zhang et al., Nucleic acids research 40.8 (2012): e55-e55 and U.S. Pub. No. 20130045508, incorporated herein by reference in their entirety. Briefly, SLiCE is a restriction site independent cloning/assembly method that is based on in vitro recombination between short regions of homologies (15-52 bp) in bacterial cell extracts derived from a RecA deficient bacterial strain engineered to contain an optimized λ prophage Red recombination system. Other recombination methods can also be used, such as recombination in yeast or phage. The cleavage products can be subject to Gibson assembly as described in, for example, Gibson et al., Nature Methods 6 (5): 343-345, and U.S. Pub. Nos. 20090275086 and 20100035768, incorporated herein by reference in their entirety. In Gibson assembly, DNA fragments containing 20-40 base pair overlap with adjacent DNA fragments are mixed with three enzymes, an exonuclease, a DNA polymerase, and a DNA ligase. In a one-tube reaction, the exonuclease creates overhangs so that adjacent DNA fragments can anneal, the DNA polymerase incorporates nucleotides to fill in any gaps, and the ligase covalently joins the DNA fragments.

One or more of the terminator oligos and primers disclosed herein can be methylated such that the terminator-attached product or amplified product can be digested with a methylation-sensitive nuclease such as MspJI, SgeI and FspEI. Such nuclease shares both type IIM and type IIS properties; thus, it only recognizes the methylation-specific 4-bp sites, ^{m}CNNR (N=A or T or C or G; R=A or G), and cuts DNA outside of this recognition sequences. Methylated primers and use thereof are disclosed in Chen et al., Nucleic Acids Research, 2013, Vol. 41, No. 8, e93, which is incorporated herein by reference in its entirety.

As will be appreciated, the compositions and systems of the disclosure are useful in various areas of biotechnology, and particularly synthetic biotechnology. For example, methods of the disclosure may be employed

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for the use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. “Consisting essentially of” means inclusion of the items listed thereafter and which is open to unlisted items that do not materially affect the basic and novel properties of the invention.

### INCORPORATION BY REFERENCE

The ASCII text file submitted herewith via EFS-Web, entitled “127662015301SequenceListing.txt” created on Dec. 21, 2016, having a size of 424 bytes, is incorporated herein by reference in its entirety.

All publications, patents and sequence database entries mentioned herein are hereby incorporated by reference in their entireties as if each individual publication or patent was specifically and individually indicated to be incorporated by reference.

1. A non-naturally occurring nucleic acid sequence comprising a Y—X—Z—O stem-loop, wherein:
a. Y is a nucleotide sequence of 5 to 30 nucleotides in length;
b. X is a nucleotide sequence of 3 to 12 nucleotides in length, each nucleotide therein not base pairing with any other nucleotide within X when Y and Z form a stem;
c. Z is a nucleotide sequence of 5 to 50 nucleotides in length and having at least 70% complementarity to Y; and
d. O is either absent or an overhang protruding from the stem formed between Y and Z.

2. The nucleic acid sequence of claim 1, wherein X forms a loop.

3. The nucleic acid sequence of claim 1, wherein O comprises a degenerate sequence.

4. The nucleic acid sequence of claim 1, comprising one or more dT-biotin.

5. The nucleic acid sequence of claim 1, wherein Y—X—Z has the sequence of SEQ ID NO.: 1.

6. A library of non-naturally occurring nucleic acid sequences, each member comprising a Y—X—Z—O stem-loop, wherein:
a. Y is a nucleotide sequence of 5 to 30 nucleotides in length;
b. X is a nucleotide sequence of 3 to 12 nucleotides in length, each nucleotide therein not base pairing with any other nucleotide within X when Y and Z form a stem;
c. Z is a nucleotide sequence of 5 to 50 nucleotides in length and having at least 70% complementarity to Y; and
d. O is an overhang protruding from the stem formed between Y and Z and comprises a degenerate sequence having N degenerate positions;
wherein the library comprises at least 4^{N} members.

7. The library of claim 6, each member comprising one or more dT-biotin.

8. The library of claim 6, wherein all members have the same Y—X—Z.

9. The library of claim 8, wherein the Y—X—Z has the sequence of SEQ ID NO.: 1.

10. A method of modifying a nucleic acid molecule, comprising attaching the nucleic acid sequence of claim 1 to the nucleic acid molecule.

11. The method of claim 11, wherein said attaching comprises ligating.

12. A method of assembling a target nucleic acid, comprising:
a. assembling a 5′ terminal construction oligonucleotide, at least one central construction oligonucleotides and a 3′ terminal construction oligonucleotide, wherein each construction oligonucleotide has two cohesive ends, at least one cohesive end being compatible with that of another construction oligonucleotide, such that when fully assembled in a predetermined order, the construction oligonucleotides form a target nucleic acid or a subconstruct thereof, wherein the 5′ terminal construction oligonucleotide has a 5′ primer binding site and the 3′ terminal construction oligonucleotide has a 3′ primer binding site;
b. attaching a terminator to an assembly product from step (a) at both ends, wherein the terminator has an overhang compatible with that of the assembly product; and
c. selectively amplifying a full assembly product using primers against the 5′ primer binding site and the 3′ primer binding site.

13. A method of assembling a target nucleic acid, comprising:
a. assembling a 5′ terminal construction oligonucleotide, at least one central construction oligonucleotides and a 3′ terminal construction oligonucleotide, wherein the at least one central construction oligonucleotides each has two cohesive ends each compatible with that of another construction oligonucleotide, such that when fully assembled in a predetermined order, the construction oligonucleotides form a target nucleic acid or a subconstruct thereof, wherein the 5′ terminal construction oligonucleotide has a 5′ blunt end and the 3′ terminal construction oligonucleotide has a 3′ blunt end;
b. attaching a terminator to a partial assembly product from step (a), wherein the terminator has an overhang compatible with that of the partial assembly product, and wherein the terminator comprises a label; and
c. removing the partial assembly product using a binding partner of the label.

14. The method of claim 13, wherein the 5′ terminal construction oligonucleotide has a 5′ primer binding site and the 3′ terminal construction oligonucleotide has a 3′ primer binding site, wherein the method further comprising amplifying a full assembly product using primers against the 5′ primer binding site and the 3′ primer binding site.

15. The method of claim 13, wherein the label is biotin and the binding partner is one or more of avidin, streptavidin and NeutrAvidin.


SEQUENCE LISTING (1 sequence)

SEQ ID NO: 1 - DNA, 60 bp, Unknown
  Synthetic sequence
gcggcggcaggagcagagcccatggaacccgagagccggcctggaccctcgggaatgaat